(19) United States
(12) Patent Application Publication
(10) Pub. No.: US 2016/0021325 A1
(43) Pub. Date: Jan. 21, 2016

(54) Procedure and Device for the Emission of Scents in Audiovisual Productions

(71) Applicant: Olorama Tecnología S.L. (LLC), Torrente, VA (ES)

(72) Inventor: Raúl Porcar, Torrente, Valencia (ES)

(21) Appl. No.: 14/407,101

(22) PCT Filed: Feb. 4, 2014
(86) PCT No.: PCT/ES2014/070076

§ 371 Date: Dec. 11, 2014

(30) Foreign Application Priority Data
Mar. 7, 2013 (ES) U 201330279

(51) Int. Cl. H04N 5/44; A61L 9/14

(52) U.S. Cl. CPC H04N 5/44; A61L 9/14

(57) ABSTRACT

A procedure for the emission of aromas/scents consists of playing, in a software, an audiovisual production with at least one encoded scent emission scene, wherein the software, for each encoded scent emission scene identified and decoded in the audiovisual production, sends a scent release signal through a wireless communication adapter (transmitter), which signal is received by a scent emission device through another wireless communication adapter (receiver). The scent emission device comprises a PCB which controls synchronized operation of a plurality of electrovalves coupled to a plurality of aerosols, for aroma/scent release, and a plurality of fan(s), to promote the a fast spread of aromas/scents, according to the encoded scent release signal received.

### FIELD OF THE INVENTION

The present invention broadly relates to the field of audiovisual productions and exhibits, such as movies, feature films, documentaries, etc. to be exhibited at movie theaters, theaters, museums, hotels etc. or homes.

Specifically, the present invention relates to computerized scent generating systems, and more particularly to computerized scent and aroma emitting systems within a networked environment or other electronic medium distribution systems or stand-alone electronic devices. In particular, the invention describes a procedure and device that improve the current, multimedia experience, through incorporating new sensations, such as smell, to those already present (i.e. sight and sound) when exhibiting audiovisual productions.

### BACKGROUND OF THE INVENTION

The goal of associating scents/odors with the images and sounds we perceive when enjoying an audiovisual production is nothing new. In 1960 Mike Todd Jr. developed the Smell-O-Vision system, a process in which scents were pumped through pipes located under individual seats. To do this, a rotating drum was designed to house bottles with the scents.

The reel for the movie Scent of a Mystery (1960) included information on when to release scents. A movie theater was adapted in New York at great expense to show the movie. This involved installing a network of small tubes behind each seat at the average height, of an audience member. The tubes were connected to a header through which the various scents were distributed. The system failed not only due to the high cost of adapting the theaters, out because the system emitted a faint high-pitched noise. What is more, the scents were not sufficiently volatile and they improperly blended into one another. The innovation never went beyond a failed effort, though it does provide an early example of the interest in incorporating scents to movies.

The above was not the only attempt. In the late 70s a rudimentary system called Odorama was developed that involved giving theater-goers cards with numbered circles on them. When specific scenes identified with these numbers were shown, the viewers were supposed to scratch and sniff the corresponding circle on the card. The movie Polyester (1981) premiered with this system.

The wide-ranging reach of the Internet has spurred a large amount of advertising on the Internet. Some companies have Internet web sites sophisticated enough to include digitized sound recordings for advertising their products. Internet users can download and play back these audio advertisements to learn more about an advertised product.

While the Internet allows advertisements via the human sense of hearing, the human sense of smell has largely been ignored on the Internet. This human sense has been ignored despite many companies having products with particular aromas or smells that may entice an Internet user to buy the products. For example, many pizza companies provide the service of ordering pizzas for home delivery via their respective Internet web sites. However, the web sites do not entice potential customers by allowing she customers to smell the pizza they are advertising.

Accordingly, it is an object of the present invention to provide the art with a computerized systems that emits smells based upon data files received over a network, such as the Internet. With such functionality, the present invention can simulate any smell ranging from fresh cut flowers or perfumes to hot, deep dish pizza. Accordingly, floral companies, perfume companies and food companies or any smell-related company can advertise through the present invention by providing a particular smell or aroma associated with their respective products.

### DESCRIPTION OF THE RELATED ART

A review of the state of the art associated with the current invention reveals several existing patents and applications covering the gamut of audiovisual productions that integrate a sense of smell for enjoyment of the viewer.

U.S. Pat. No. 4,623,604, issued on 16 Dec. 1986 to Spector and titled “Multi-Aroma Cartridge Player”, describes an aroma dispenser comprising a container with a series of compartments containing various scent-impregnated pads, which container is inserted into a playback device having a series of associated heating elements adjoining the compartments, to heat them for selective release of odors, either manually or automated in synchronization with the displayed scenes of an audiovisual production, such as a video or movie. A variety of scents is emitted as various heating elements are activated to evaporate certain scents in the slots with the impregnated pads in the corresponding compartment. The use of this device is imprecise, as it is slow to activate and synchronization with the audiovisual production being played is difficult.

Likewise, U.S. Pat. No. 4,905,112, issued to Rhodes on 27 Feb. 1990, titled “Scent cassette”, discloses a tape cassette with a perforated housing having a compartment that holds a material with the scent or odor to be emitted. Air is blown across the tape at all times to release the scent to the environment in synchronization with the audiovisual production being played. The device does not allow digital processing of the scent emitting scenes or their synchronization with the audiovisual content of the production.

As can be seen, such a device has the disadvantage that its use requires a recorded medium, be it a cassette, CD, DVD, etc., as well as a playback device for the recording that is especially adapted to trigger the emission of scents or odors during playback of the recording based on inaudible and/or invisible signals that are pre-recorded along with the sound and image on the audiovisual product, signals whose implementation is unknown.

U.S. Pat. No. 5,069,876, issued on 3 Dec. 1991 to Oshinsky, titled “Combined Scent and Audio Point of Sale Display Unit”, describes a device with one or several scent cartridges in combination with an audio tape. Only one scent can be emitted at a time with this device.

U.S. Pat. No. 5,565,148, issued on 15 Oct. 1996 to Pendergrass, titled “Device for Selectively Providing a Multiplicity of Aromas”, describes a device with a plurality of separate cylinders, each of which houses a scent-emitting material. The cylinders are arranged concentrically around the central axis of the cylinder. The cylinder is rotated into the desired position and a source blows across volatile scent cartridge to release the scent from the device. This device can only emit one scent at a time.

U.S. Pat. No. 5,949,522, issued 7 Sep. 1999 to Manne, for “Precision Fragrance Dispenser Apparatus”, describes a system using tubes to deliver the scent or scents to a user's nose instead of to the ambient air. It offers no way of synchronizing the delivery to the audiovisual content.

PCT Patent No. 99/38,102 issued on 29 Jul. 1999 to Bala Harayanaswamy, titled “Olfactory Emitting System” describes a system in which one or more pressurized scented gasses are released when certain preset words or phrases trigger a voice recognition system to actuate valves. This system is intended to release scents at a remote point.

Japanese patent no. 2003/260122, issued to Nagasaka Hideo (Applicant: SONY CORP), describes a method and device for releasing scents when a musical instrument is played.

U.S. Pat. No. 6,542,442, issued on 6 Dec. 2001 to Kaslon, generically relating to a single device that emits one or more scents, describes a scent-emitting device incorporated into a specially adapted recording medium (videocassette or DVD or other pre-recorded media) and into special a playback device for reproducing the recording on said pre-recorded media, the scent-emitting device triggered by the input that is also adapted to provide olfactory stimulation. Recordings include one or more scent emitting cartridges in the medium containing the audiovisual information itself and the playback device includes a scent dispersal apparatus. In addition, the recording includes inaudible and/or invisible signals which trigger the scent emitting device to release the scents upon receiving an appropriate signal is from the recording, which actuates a small pump that releases a small amount of desired scent from the corresponding cartridge. This scent is sprayed into a pressurized chamber, from which it is vaporized and dispersed into the ambient air by a fan or other suitable device. Each pre-recorded cassette may include one or more scent emitting cartridges, and the player includes a series of syringes that automatically penetrate each cartridge on the cassette as it is inserted into the player. Implementation of the ‘scent signals’ is not specified.

Similarities with US Patent Application 2005/195367 A1 by Selander et al, published Sep. 8, 2003 are based on a procedure of encoding and decoding a scent track. US Patent Application 2003/026587 A1 by Ostrover, published Feb. 6, 2003 also describes an encoding system. However, the present invention does not include a parallel scent track as part of the main audiovisual media stream. This is contained instead in a separate file which may be updated remotely and “behind the scenes” and is completely transparent to the end-user, who need not acquire a special audiovisual production encoded for scents. Therefore, the superior system design of the present invention is a remote paradigm, wherein the scent features of a production may be improved without user action, controlling cartridge consumption, error detection and problem solving.

In neither of the aforementioned too applications is the user able to merely connect a small device (such as a PCB) to a Bluray Disc player (or other player) and play. Rather, they require special extraneous software to run. The present solution allows for operating the hardware as a Direct Motion Device”, connected to a PCB directly connected to a BD player.

US Patent Application 2003/026728 A1 by Avram. published on Feb. 6, 2003, relates to substance generation in multi-media presentations, with special effects including climatic and environmental simulation, as well as containment and evacuation of contaminated mediums in indoor or outdoor spaces and crowd control. It accordingly utilizes aromatizers and discloses a solution based on aerosols and fans. However, wireless capability, scale and size are the key differences here. Avram requires huge equipment to produce 12 aromas, whereas the immediate invention reproduces the same capability in dimensions of 24×17×11 cm and by a wireless method, which is not offered by Avram. Thus installation of the present technology solution is infinitely simpler than Avram, making in horns installation possible, unlike Avram, due to huge equipment requirements.

To summarize, none of the last three applications may be used at home (due to big equipment requirements), none may be updated remotely, adding scents to movies remotely behind the scenes, none of them works directly with Bluray or DVD players, none work wirelessly without cables (they all need cables at least for power, where the current invention does not), and none offers a tablet or portable solution to manually activate scents, the entire solution in a very small wireless device of just 24×17×11 cm.

A simple and affordable way is thus needed to modify known technologies for emitting scents/odors in audiovisual products to overcome the problems described above; mainly, the need to employ complex recording media and video playback devices adapted to these purposes, as well as the difficulty in synchronizing the emission of the scents/odors with the sounds and images on the audiovisual product.

### SUMMARY OF THE INVENTION

In accordance with an aspect of the present invention, a scent data structure is utilized for containing scent data indicative of a scent that is to be emitted. A first computer that is connected to a network receives the scent data structure over the network. A scent emission device that is connected to the first computer and to a propellant and diffusion mechanism controls the emission of scents from their containers based upon the received scent data. In accordance with a second aspect of the invention, a method for emitting a scent within a computerized network environment includes generating scent data indicative of the scent to be emitted. The scent data structure is transmitted across the network and is received at a first computer. The emission of a scent in a predetermined amount from its container is controlled based upon the received scent data.

Additional objects and advantages of the present invention will be apparent from the detailed description of the preferred embodiment, the appended claims in the accompanying drawings, ox may be learned by practice of the invention.

As mentioned, an objective of the present invention is to set out a procedure for emission of scents in audiovisual productions comprising the following steps:
a) to play an audiovisual production with at least one scene with scents encoded, using a Video Software player developed to identify and decode the scenes associated with particular scents,
b) identify and decode the scenes with scents as stated in step a),
c) transmit a signal for delivery of scents by means of a wirelessly-controlled scent-emitting device using a wireless adapter (transmitter),
d) receive the signal sent in step c) by a wirelessly controlled scent-emitting device (WSED) through a wireless adapter (receiver),
e) decode the signal received in step d),
f) transmit this signal decoded in step e) to a scent-emission controller and air-flow controller
respectively,
g) send an activation signal to one (or several) electrovalves of a set of aerosols for scent release, according to the signal sent by the controller specified in step f) and
h) activate one (or several) fans, according to the signal sent by the air flow controller.

furthermore, it is also an objective of the present invention to create a device and apparatus for the emission of scents in audiovisual products, which is used in steps d) to h) of the procedure described.

The device is composed of a set of aerosol cans with scents and propellant gas. Cans are tied to electrovalves (EV) which control the emission of scents.

Either aerosol cans or electrovalves are mounted on a base structure (chassis), and at least 1 fan is installed behind the scent flow, providing for easier and faster distribution of scents.

Electrovalves and fan(s) are connected to a scent emitter and an air flow controller respectively. These controllers are integrated into a circuit or printed circuit board (PCB) which controls the action of the electrovalves and fan(s) according to the scent-release signal received through special wireless adapters also integrated into the PCB.

As shown, this solution improves and simplifies other technologies for the emission of scents in audiovisual products. It is successfully achieved a synchronization of the video player and the scent-emitting device, allowing for wireless operation of the latter, and thus, its capability to be installed at the most suitable location, regardless of the position of the player, either in public spaces (theaters, museums, hotels, etc.) or in home theaters (or homes).

Furthermore, information on scents integrated into an audiovisual production may be incorporated prior to production or thereafter, retaining multi-compatibility of a production, which may still be played in standard players without an OLORAMA™ decoder.

### BRIEF DESCRIPTION OF THE DRAWINGS

The present invention is illustrated with a set of drawings, which not in any way be seen as limiting the scope of the invention:

FIG. 1 shows a scheme of the internal view of the device for emission of scents in audiovisual productions.

FIG. 2 shows a scheme of the side view of aerosols, and corresponding electrovalves, also present in FIG. 1.

FIG. 3 shows a scheme of the front view of the first implementation of the base structure or chassis that holds the aerosols/valves present in FIG. 1.

FIG. 4 shows a top view of the first implementation of the base structure shown in FIG. 3.

FIG. 5 shows a top view of a second implementation of the base structure which holds aerosol/valves shown in FIG. 1,

The other numbers included in the drawings are a key as specified below:
(1) Printed Circuit Board (PCB)
(2) Wireless receivers
(3) Batteries
(4) External connector
(5) Set of aerosols
(6) Electrovalves (1 per aerosol)
(7) Fan(s)
(8) Base structure to arrange aerosols/valves (2 implementations, one for a wirelessly controlled aromatizer and second for a high power diffuser)
(9) Air flow controller
(10) Scent-emission controller
(11) Signal decoder

### DETAILED DESCRIPTION OF THE PREFERRED EMBODIMENTS

The procedure for the emission of aromas/scents in audiovisual products according to the present invention is implemented by playing an audiovisual product with at least one scene incorporating scent emission encoded by a video player software capable of decoding this scene and emitting the desired corresponding scent.

As shown, before playing the audiovisual production in the video player, at least one scene must be encoded for scent emission within the audiovisual production. To achieve that it is preferable to play the audiovisual production in authoring software designed to encode scenes coupled with scents in such productions. Once a scene of interest is identified, play is stopped and the software lets the user encode a scent emission according to the scene.

Either playing the audiovisual production, or stopping in scenes of interest and decoding a scent emission according to the scene could be achieved repetitively (where such scenes of interest exist in the audiovisual production).

Finally, the authoring software creates a new archive which contains the information regarding scent emission. This archive may be named as an SDA (Scent Description Archive) (*.sda), whose preferred storage format is XML. This archive is independent of the original audiovisual archive (thus it is not included as an extra track), but synchronized with it, so maintaining compatibility of the original audiovisual archive.

It is also preferable that this file be saved with encryption, using a dynamic keys encryption system. Thus a stronger protection is achieved as is allowed in order to invalidate already cracked keys using new keys.

In addition, the authoring software is preferred to he constructed based on a service-oriented architecture (SOA), which allows changes to be made without rebuilding all the Software. In particular, changes could affect three modules:
1. A module to build an SDA archive.
2. A module inherits and incorporates the SDA archive to the audiovisual product archive.
3. An encryption module.

In one example of a preferred embodiment of encoding at least one scene with scents in an audiovisual production, a video player is required to decode the information and play an audiovisual production with scents. During the play, such a player identifies and decodes scenes incorporating emission of scents, sending a scent release signal through a wireless adapter (transmitter).

Such a signal relays information containing the scent to be released and the duration of the emission.

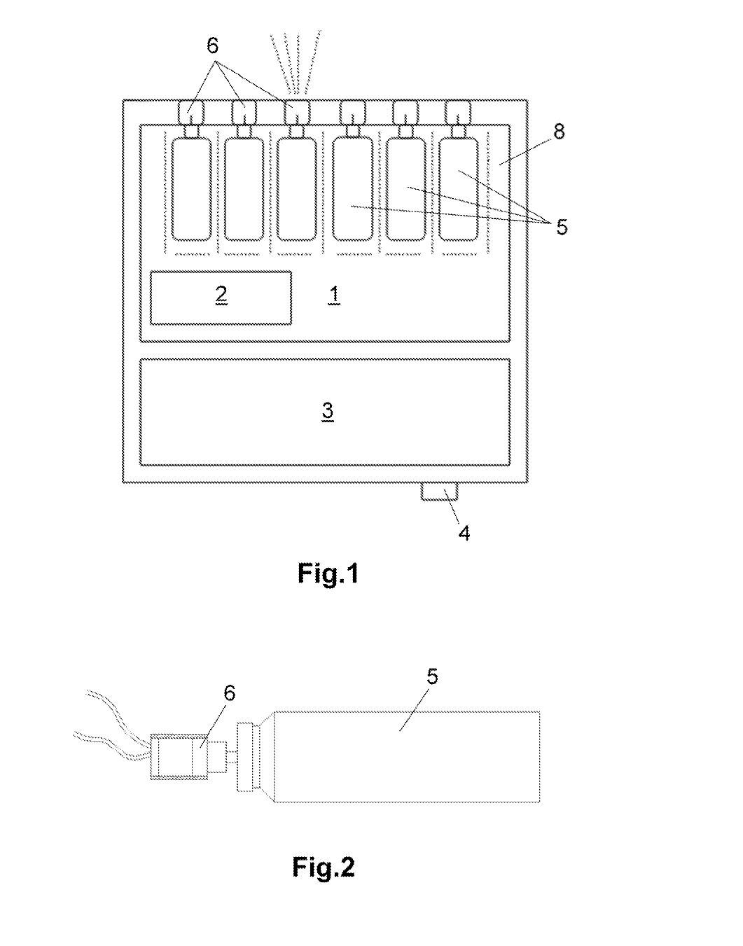

Once this signal of scent emission is transmitted, it is received by the wireless adapters (receivers) of an aroma/scent emitter device, shown in FIGS. 1 to 6. Such emission signal received is decoded and then retransmitted to an aroma/scent emission controller (10) and a fan activation controller (9) respectively.

Then, an activation signal is transmitted by the emitter to one or many electrovalves (6) of a set of aerosols (5), for emission of aromas/scents, according to a scent release signal transmitted to the emission controllers (10), whilst one or many fans are activated (7), according to a fan activation signal received in a fan activation controller (9).

It is preferable, during playing of the audiovisual production in the player software, when a scene with scent emission is identified and decoded, that a scent emission signal be transmitted, which shall then be received by the aroma/scent emitting device, as described and shown in FIGS. 1 to 6, and decoded for transmission respectively to the scent emission controllers (10) and fan activation controller (9), which shall send activation signals to electrovalve(s) (6) and fan(s) (7), which shall then respectively operate according to the information encoded within the audiovisual production.

Likewise, video player software is preferred wherein:

an audiovisual production is played with at least one scene with scents,

such a scent emission scene is identified and decoded, and

an encoded signal for scent emission is sent based on this scene

It is preferable that this video player runs on a PC, or electronic device such as a Tablet, Smart-phone, Smart-TV, etc.

Furthermore, the aroma/scent emitting device that receives the scent emission signal encoded through wireless communication devices (2), for example, via a Wi-Fi protocol, contains at least one set of aerosol cans (5) that contain aromas/scents and a gas-based propellant.

As shown in FIG. 2, the aerosols (5) are coupled to electrovalves (6) which handle the scent emission. Preferably these shall be implemented as solenoid valves.

As shown in FIG. 1, 3, 4 and 5, both aerosols (5) and electrovalves (6) are assembled to a base structure (8), and at least one fan (7) is assembled behind (5) them, to achieve a proper spread or aromas/scents in the air.

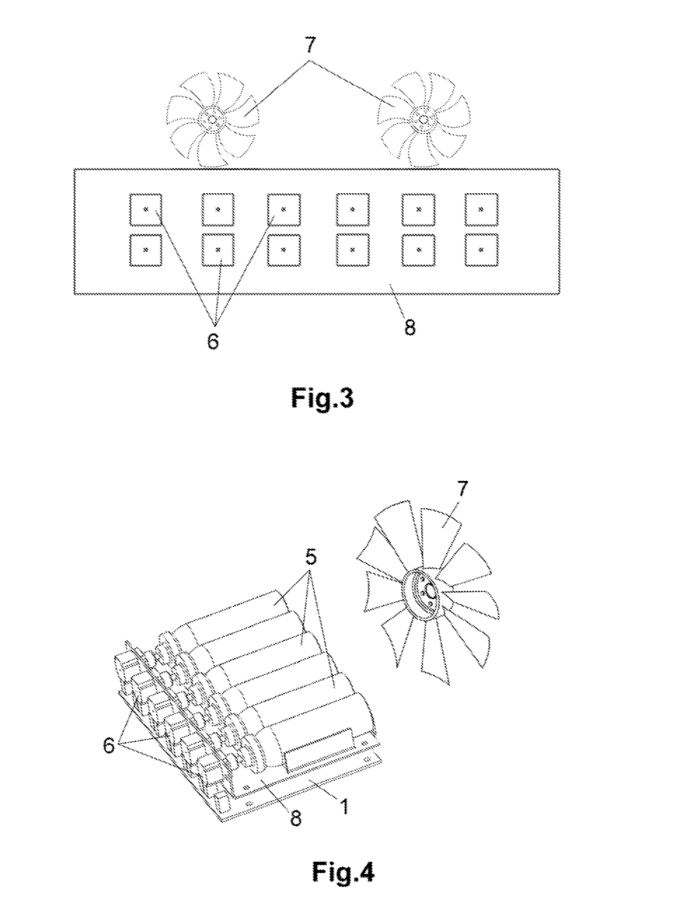

In a first embodiment, shown in FIGS. 3 and 4, the base structure (8) is a simple platform where aerosols (5) are arranged, with corresponding electrovalves (6), in a straight sequential arrangement on top of the basic structure, spaced equidistantly one after another. In this particular case it is preferable to locate the fans (7) above the base structure.

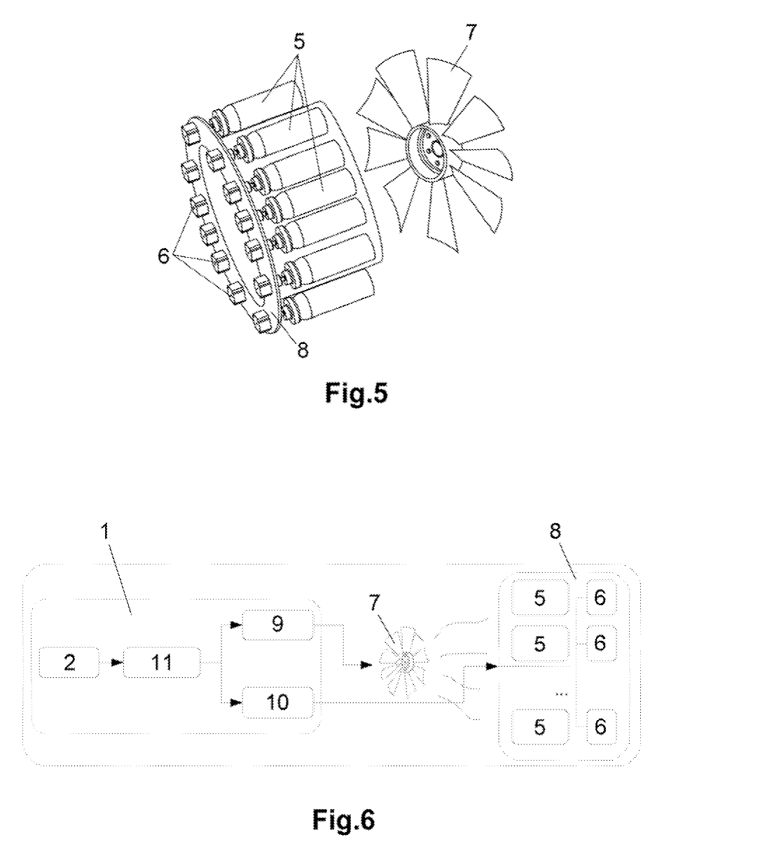

FIG. 5 displays a second example of an embodiment, wherein the base structure (8) is configured as a pipe, i.e. cylindrically, assembling aerosols (5) with their respective electrovalves (6) in a circular arrangement surrounding the structure (8). In this case the fan could be assembled behind the base structure.

As shown in FIG. 6, electrovalves (6) and fans (7) are connected to a scent emission controller (9) and a fan activation controller (10) respectively. Controllers (9, 10) are integrated in a PCB (Printed Circuit Board) that handles the synchronous operation of electrovalves (6) and fans (7), according to the scent emission signal received through the wireless communication adapters (2) integrated in the PCB (1).

Preferably the PCB (1) shall contain a signal decoder (11), to decode the scent emission signal received through the wireless communication adapters (2) integrated in the PCB (1).

Likewise, preferably the scent emitter device shall contain at least one battery (3) that provides power to PCB (1) and fan(s) (7), for automatic operation. The battery (3) incorporates an external connector (4), which may be connected to and recharged by a wall supply system (not shown).

To support longer operating periods, the device integrates components having low power consumption.

As shown, either flexibility in power supply (through chargeable batteries) of the PCB and the fan(s), and wireless communication allow for very simple installation of the aroma/scent emitter in almost any place, either private (home) or public (theaters, museums, hotels, etc.), and a Bluray Disc player may be directly connected to a BD player which in turn connects to a PCB which controls the hardware as a “Direct Motion Device”.

1. A procedure for emission of aromas and scents in audiovisual productions comprising the following steps:
a) playing an audiovisual production with at least one scene involving emission of scents encoded in a software video player capable of identifying and decoding the scent emission scene;
b) identifying and decoding the scent emission scene;
c) sending an encoded scent release signal through wireless communication adapters;
d) receiving the encoded, scent release signal by an aroma and scent emission device through wireless communication adapters;
e) decoding the scent release signal received by the aroma and scent emission device;
f) transmitting the decoded signal to an aroma and scent controller and to a fan controller respectively.
g) activating a plurality of electrovalves of a set of aerosols for the release of aromas and scents, according to the transmitted signal; and
h) activating a plurality of fans, according to the transmitted signal.

2. The procedure of claim 1 further comprising, prior to the initial step of playing the audiovisual production, the step of encoding at least one scent emission scene into the audiovisual production further comprising the following steps:
i) playing the audiovisual production in an authoring software capable of encoding scent emission scenes in the audiovisual production;
ii) stopping the play of the audiovisual product in one scene to encode the scent emission scene;
iii) encoding scent emission scenes into the audiovisual production;
iv) saving the audiovisual production with at least one scent omission scene encoded;

3. The procedure of claim 2 wherein the steps are repeated while the audiovisual production contains scenes of interest for scent emission.

4. The procedure of claim 1 wherein steps all the steps are repeated when scent emission scenes are identified and decoded in the audiovisual production being played.

5. Procedure or claim 1 wherein by means of the encoded scent emission scene and the scent release signal, information is transmitted concerning aromas and scents to be released, at a specific instant, with a specific intensity and for a specific duration.

6. The procedure of claim. 1 wherein the software video player runs on a PC, a tablet, a smartphones or a smart-TV.

7. A device for the emission of aromas and scents in audiovisual productions, for use by the steps of claim 1, comprising a plurality of aerosols containing aromas and scents diffused in a propellant gas and a plurality of electrovalves controlling emission of aromas and scents, both of which are assembled on a base structure, and a plurality of fans located next to the aerosols to promote the easy spread and diffusion of aromas and scents into the ambient atmosphere wherein the electrovalves and fans are connected, with and controlled by an aroma and scent emission controller as well as a fan activation controller respectively, the controllers being integrated into a printed circuit board which controls the synchronized operating of the electrovalves and fans according to a scent emission signal encoded and received through wireless receivers also integrated into the printed circuit board.

8. The device of claim 7 wherein the PCB (1) contains a signal decoder (11) that decodes this signal in the wireless media receptors (2) and It is transmitted simultaneously to the scent emission controller (9) and fan activation controller (10).

9. The device of claim 7 wherein the electrovalves are chosen from the family of solenoid valves

10. The device of claim 7 wherein the base structure is planar, with the plurality of aerosols the corresponding plurality of electrovalves are aligned over this structure.

11. The device of claim 7 wherein the plurality of fans is disposed above the basic structure.

12. The device of claim 7 wherein the base structure is configured as a tube or cylinder, with the aerosols and their corresponding electrovalves disposed following a circular path surrounding the structure.

13. The device of claim 7 wherein the plurality of fans is disposed behind the structure.

14. The device of claim 7 further comprising at least one battery which supplies power to the PCB and the fans for an automatic operation of the device.
